(19) United States
(12) Patent Application Publication
(10) Pub. No.: US 2009/0068161 A1
(43) Pub. Date: Mar. 12, 2009

(54) USE OF A COMBINATION OF HESPERIDIN AND OF A MICROORGANISM FOR INFLUENCING THE BARRIER FUNCTION OF THE SKIN

(75) Inventors: Audrey GUENICHE, Rueil Malmaison (FR); Isabelle CASTIEL, Nice (FR)

Correspondence Address: OLIFF & BERRIDGE, PLC, P.O. BOX 320850, ALEXANDRIA, VA, 22320-4850, US

(73) Assignees: L'OREAL, Paris (FR); NESTEC S.A., Vevey (CH)

(21) Appl. No.: 12/204,437

(22) Filed: Sep. 4, 2008

Related U.S. Application Data
(60) Provisional application No. 60/960,221, filed on Sep. 20, 2007.
(60) Provisional application No. 60/960,224, filed on Sep. 20, 2007.

(30) Foreign Application Priority Data
Sep. 4, 2007 (FR) 07 57346
Sep. 4, 2007 (FR) 07 57350

(51) Int. Cl. A61K 35/74; A61K 35/66; A61K 31/7028; A61Q 17/00; A61K 8/99; A61K 36/062

(52) U.S. Cl. 424/93.42; 424/93.1; 514/25; 424/195.16; 424/93.45; 424/93.44; 424/93.46

(57) ABSTRACT

The cosmetic use of at least an effective amount of hesperidin or of one of its derivatives in combination with at least an effective amount of at least one microorganism, in particular probiotic microorganism, or one of its fractions as agent for preventing a reduction in and/or for reinforcing the barrier function of the skin.

This non provisional application claims the benefit of French Applications No. 07 57346 and No. 07 57350 filed on Sep. 4, 2007 and U.S. Provisional Applications No. 60/960,221 and No. 60/960,224 filed on Sep. 20, 2007.

The present disclosure relates to the use, in particular cosmetic use, of a combination, the said combination being intended to prevent a reduction in and/or to reinforce the barrier function of the skin and in particular to prevent and/or treat the associated disorders, especially those due to cutaneous dryness.

The present disclosure also relates to the prevention and/or treatment of cutaneous signs of ageing and/or photoageing, in particular those induced or exacerbated by pollution.

### BACKGROUND

The human skin is composed of two compartments, namely a deep compartment, the dermis, and a surface compartment, the epidermis.

It constitutes a barrier against external attacks, in particular chemical, mechanical or infectious attacks, and, therefore, a number of defensive reactions against environmental factors (climate, ultraviolet rays, tobacco, and the like) and/or xenobiotic, such as, for example, microorganisms, occur therein. This property, referred to as barrier function, is mainly provided by the most superficial layer of the epidermis, namely the horny layer, referred to as the stratum corneum.

The cells constituting the epidermis (predominantly keratinocytes but also melanocytes and Langerhans cells) are delimited by an intercellular lipid structure. Each of these cell types contributes, via its own functions, to the essential role played by the skin in the organism. In particular, the keratinocytes undergo a process of continuous and oriented maturation which, from the keratinocytes which are in the basal layer of the epidermis, results in the formation of corneocytes, which are completely keratinized dead cells composed of keratinocytes in the terminal stage of their differentiation.

During differentiation, the phospholipids, the role of which consists in producing the fluid structure of the cell membranes of the living layers of the epidermis, are gradually replaced by a mixture composed predominantly of fatty acids, of cholesterol and of sphingolipids (ceramides). These lipids, which are organized in specific lamellar liquid crystal phases, form the intracellular cement of the stratum corneum and are essential for the water exchanges and the barrier function of the epidermis. Thus, the lamellar structure of the lipids of the lipid domain of the epidermis and the corneocytes participate in the epidermal barrier function.

It is clear that the quality of the cutaneous barrier and of the mucous membranes depends on complex endogenous biological mechanisms involving many growth factors, adhesion molecules, hormones and lipid metabolism enzymes.

Thus, a detrimental change in the cutaneous barrier can occur in the presence of external attacks, such as irritants (detergents, acids, bases, oxidizing agents, reducing agents, concentrated solvents, toxic gases or fumes), mechanical stresses (rubbing, impacts, abrasion, tearing of the surface, projection of dust or particles, shaving or depilation), heat or climatic imbalances (cold, dryness, radiation) or xenobiotics (undesirable microorganisms, allergens), or of internal attacks, such as psychological stress.

This detrimental change in the cutaneous barrier can be reflected in particular by cutaneous discomfort, sensory phenomena and in particular unpleasant phenomena, or also cutaneous dryness, which can in particular be measured by the imperceptible water loss. A person may then experience a feeling of cutaneous discomfort which may be symptomized in particular by smarting, tightness, a feeling of tautness, inflammation and/or itching.

These feelings of cutaneous discomfort are more frequent in the most exposed areas of the body, namely the hands, feet, face and scalp.

They can in particular occur on areas subjected to certain daily or frequently repeated acts of hygiene, such as shaving, depilation, cleaning with toiletries or household products, the application of adhesives (dressings, patches, attachment of prostheses) or in the case of sporting or occupational acts, or acts related simply to lifestyle and to the use of clothing, tools or equipment which generate localized friction. They can also be enhanced by psychological stress.

Feelings of cutaneous discomfort, sensorial phenomena or cutaneous dryness affect individuals having any type of skin, normal and even greasy, and very particularly:
individuals with “fragile” or “delicate” skin which is vulnerable to external factors, which is often accompanied by erythema and rosacea, and which rapidly becomes unbalanced, for example during large variations in temperature or relative humidity (in the case of the skin of babies, for example);
individuals with “weakened” skin, which groups together in particular:
   individuals for whom cutaneous metabolism declines and very particularly for whom the protective aqueous/lipid film composed of sweat, sebum and natural moisturizing factors is in short supply, as is the case for individuals aged more than 60 years and in particular in the context of great age (at least 75 years). Skin of these types is described as “senile skin”;
   individuals for whom the composition of the aqueous/lipid film is modified, as is the case for diabetic individuals, individuals undergoing dialysis or individuals affected by certain diseases such as xerosis vulgaris (of probable genetic origin and being manifested predominantly on the face, the limbs and the back of the hands).

Reference may also be made to individuals with “abused” skin, for example for skin which has been shaved.

The aim is thus to prevent a reduction in and/or to reinforce the cutaneous barrier function in order:
to prevent and/or reduce feelings of cutaneous discomfort, of smarting, tightness, inflammation and itching, in particular in individuals with fragile or delicate skin (for example babies); or individuals with weakened skin (such as individuals aged at least 60 years and in particular individuals of at least 75 years), or individuals for whom the composition of the aqueous/lipid film is modified, as is the case for diabetic individuals, individuals undergoing dialysis or individuals affected by certain diseases,
and/or to improve the cutaneous barrier function of skin affected by atopy and/or to prolong the phases of remission between acute crises of this type of condition.

Another aim is to prevent a reduction in and/or to reinforce the cutaneous barrier function in order:
to treat cutaneous dryness states, squamous states; in particular dandruff states;
to treat dry skin, in particular hyposeborrhoeic dry skin;
to treat itching and/or tightness associated with dry skin;
to treat cutaneous disorders related to a deficiency of excretion and/or of secretion of sebum;
to physiologically restore an appropriate state of hydration to the stratum corneum;
to treat dry keratinous fibres;
to treat functional disorders of the pilosebaceous unit;
to prevent and/or reduce wrinkles related to cutaneous dryness;
to improve the comfort of dry skin and a dry scalp;
to combat the dull and/or lifeless appearance of the skin and/or hair as a consequence of it drying.

As regards more particularly cutaneous dryness, it is manifested essentially by a feeling of tightness and/or of tautness. When a skin suffers from dryness, it is also rough to the touch and appears covered with squamae. When cutaneous dryness is slight, these squamae are profuse but not very visible to the naked eye. They become less numerous but increasingly visible to the naked eye when this disorder worsens.

Cutaneous dryness can also be associated with a fall in the level of hydration of the cutaneous barrier and can in particular be evaluated by corneometry.

The origin of this cutaneous dryness can be of constitutional or acquired type.

Furthermore, over time, various signs appear on the skin which are highly characteristic of intrinsic ageing, being reflected in particular by a modification of the cutaneous structure and functions.

Another component of ageing is of exogenous origin (Yaar and Gilchrest, J. Invest. Dermatol., 1998). This is because ageing can be accelerated by environmental factors, such as repeated exposure of the skin to sunlight, in particular to ultraviolet A and B radiation, or to pollution. Thus, various types of chemicals, xenobiotics and particles are the components of urban pollution. Among these compounds, three main categories of pollutants can exert detrimental effects on the skin: gases, heavy metals and particles, which are the combustion residues on which a great many organic compounds are adsorbed.

What is more, in urban pollution, simultaneous exposure to O₃ and to UV radiation can cause synergistic oxidative stress.

Likewise, it may be supposed that there exists a synergy of action between ozone and organic compounds resulting from combustion.

### SUMMARY

For obvious reasons, there is thus a continuous search to improve the resistance of the skin to gases, heavy metals, organic compounds which are combustion residues, and their detrimental effects (maximized by UV radiation) encountered in particular in urban pollution, acting in isolation or in combination, and in fact to slow down signs of irritation, ageing and/or photoageing induced in particular by detrimental tissue change induced by the said pollutants.

Consequently, the use of substances which would have the ability to protect the cells of the skin and the extracellular matrix with regard to the abovementioned attacks might also reduce the signs and detrimental changes related to ageing and/or to photoageing, in particular those induced or exacerbated by pollution.

In this context, the inventors have discovered that the combination of hesperidin or of one of its derivatives and of at least one microorganism, in particular probiotic microorganism, or of one of its fractions is capable of preventing a reduction in and/or of reinforcing the barrier function of the skin.

In particular, they have demonstrated that such a combination proves to be particularly effective in preventing and/or treating cutaneous dryness states and in particular acquired and/or constitutional cutaneous dryness states.

Such a finding is based on the observation, by the inventors, of an effectiveness of the combination under consideration in treating dryness of keratinous substances and associated disorders.

In particular, the inventors have discovered that the combination under consideration according to the disclosure may make it possible to prevent and/or significantly limit dehydration of the skin.

Thus, they have more specifically demonstrated that such a combination proves to be particularly effective in preventing and/or treating dry skin and more particularly still acquired dry skin and/or constitutional dry skin.

In the case of acquired cutaneous dryness, the involvement of external parameters, such as exposure to chemical agents, to difficult climatic conditions or to solar radiation or alternatively some therapeutic treatments (for example retinoids), is determining. Under these external influences, the skin can then become temporarily and locally dry.

In the case of constitutional cutaneous dryness, it is possible to distinguish two categories: pathologic and nonpathologic cutaneous dryness.

Pathologic constitutional cutaneous dryness is essentially represented by atopic dermatitis and ichthyosis. It is virtually independent of the external conditions.

Atopic dermatitis is described as associated with a deficiency in the metabolism of the lipids of the stratum corneum and in particular of the ceramides. This pathology presents itself in the form of a more or less chronic xerosis effecting a wide expanse of the body, associated with inflammatory and pruriginous eruptions by patches.

Ichthyosis are pathologies characterized by a genetic deficiency affecting the keratinization process at various stages. It is manifested by significant desquamation by patches.

Nonpathologic constitutional cutaneous dryness characterizes dry skin, the severity of which can depend on the external factors already mentioned.

The use of flavonoids, including hesperidin, to increase cell proliferation and to treat in particular scars is already known from WO 03/057210.

Furthermore, WO 2005/058255 describes the action of specific flavanones, including hesperidin, for treating disorders of the skin and hair, in particular via the cytoprotective and antiinflammatory properties of the latter.

FR 2 802 088 and DE 1 980 6890 describe, for their part, the properties of a citrus extract dosed with hesperidin in order to re-establish or maintain the radiance of the skin or hair.

Furthermore, it is known to employ microorganisms for caring for and/or treating keratinous substances.

The document WO 02/28402 discloses the use of probiotic microorganisms for regulating cutaneous hypersensitivity reactions, such as inflammatory and allergic reactions, which come under an inflammatory process.

WO 03/070260 for its part describes that such microorganisms can be used for skin photoprotective purposes.

However, the combination of hesperidin with a microorganism is never described therein.

EP 0 774 249 discloses the effect of a combination of specific flavanones, on the one hand, and of a combination of specific flavanones with a specific ceramide, on the other hand, on the differentiation of keratinocytes.

The document WO 2006/037 922 for its part is targeted at compositions intended for the treatment of sensitive skin, which employ a combination of two microorganisms.

As regards FR 2 872 047, it describes the combination of a probiotic micro-organism with a divalent inorganic cation.

Finally, FR 2 889 057 discloses a topical composition comprising a micro-organism in combination with a polyunsaturated fatty acid and/or polyunsaturated fatty acid ester of use in the treatment of sensitive skin.

The combination of an effective amount of hesperidin or of one of its derivatives and of an effective amount of at least one microorganism, in particular probiotic microorganism, or of one of its fractions has thus never, until now, been used to prevent a reduction in and/or to reinforce the barrier function of the skin and in particular to prevent and/or treat disorders associated with cutaneous dryness.

Due to the prevention of a reduction and/or the reinforcing of the cutaneous barrier function brought about by the administration of a combination according to the disclosure, all skin types and in particular fragile or weakened skin (for example the skin of babies, of individuals of at least 60 years, preferably of at least 75 years, of diabetic individuals or individuals undergoing dialysis) or skin suffering from dryness are better protected from chemical, mechanical or infectious external attacks.

The Inventors have in particular demonstrated, on a reconstituted skin model, an improvement in the barrier function of the skin after treatment of the said skin with the combination according to the disclosure.

The disclosure thus relates, according to a first of its aspects, to the cosmetic use of at least an effective amount of hesperidin or of one of its derivatives in combination with at least an effective amount of a least one microorganism, in particular probiotic microorganism, or one of its fractions as agent for preventing a reduction in and/or reinforcing the barrier function of the skin, in particular as agent for preventing and/or treating disorders associated with cutaneous dryness.

It also relates to the cosmetic use of an effective amount of hesperidin or one of its derivatives in combination with at least an effective amount of at least one micro-organism, in particular probiotic microorganism, or of one of its fractions as agent for preventing and/or treating cutaneous dryness states.

It also relates to the cosmetic use of an effective amount of hesperidin or of one of its derivatives in combination with at least an effective amount of at least one microorganism, in particular probiotic microorganism, or of one of its fractions as agent for preventing and/or treating dry keratinous substances and in particular dry skin, and such being the case associated disorders thereof.

It is also targeted at the use of an effective amount of hesperidin or of one of its derivatives in combination with at least an effective amount of at least one micro-organism, in particular probiotic microorganism, or of one of its fractions in the preparation of a composition, in particular cosmetic and/or dermatological composition, intended to prevent and/or treat cutaneous dryness states and in particular acquired and/or constitutional cutaneous dryness.

It also relates to the use of an effective amount of hesperidin or of one of its derivatives in combination with at least an effective amount of at least one microorganism, in particular probiotic microorganism, or of one of its fractions in the preparation of a composition, in particular cosmetic and/or dermatological composition, intended to prevent and/or treat dry keratinous substances and in particular dry skin and very particularly acquired dry skin and/or constitutional dry skin.

When the keratinous substances are human or animal keratinous fibres, such as the hair, body hairs and/or eyelashes, the combination according to the disclosure proves to be particularly advantageous in preventing and/or treating the expression of signs of weakness, such as, for example, dryness, which is generally reflected by a brittle aspect of the fibre.

The combination according to the disclosure can thus make it possible to confer a glossy appearance on keratinous fibres, in particular on human hair and on the coats of animals.

The combination under consideration according to the disclosure thus makes it possible to provide for the maintenance of the barrier function of the skin at its level of normal effectiveness, that is to say the level at which it provides its function of protecting the body.

Within the meaning of the disclosure, the expression “to reinforce the barrier function of the skin” means to improve the barrier function of the skin.

This improvement is in particular determining when the barrier function of the skin is detrimentally affected and when it is necessary to reestablish it. This detrimental change in the skin can be due in particular to a state of dryness of the keratinous substances and in particular cutaneous dryness.

It can also be advantageous when it is desired to strengthen the native barrier function of the skin in order in particular to confer on the body better resistance to external attacks to which it is liable to be exposed.

The disclosure also relates, according to another of its aspects, to the cosmetic use of at least an effective amount of hesperidin or of one of its derivatives in combination with at least an effective amount of at least one microorganism, in particular probiotic microorganism, or one of its fractions as agent for reinforcing the protection of the skin with regard to external attacks.

In particular, the said combination and/or a composition comprising such a combination can be intended to prevent and/or reduce cutaneous discomfort of the skin brought about in particular by an exogenous stress of chemical, environmental or mechanical origin and/or an endogenous stress, in particular of fragile and/or weakened skin and/or skin suffering from dryness, as defined above.

The cutaneous discomfort can in particular be characterized by tightness, smarting, inflammation and/or itching.

According to another embodiment, the said combination and/or a composition comprising such a combination can be intended to prevent a reduction in and/or to reinforce the barrier function of skin chosen from fragile skin, especially atopic skin, weakened skin, abused skin and/or skin suffering from dryness.

In the context of the disclosure, the combination according to the disclosure can be used for application to healthy skin subjected to or which may be subjected to the influence of agents such as climatic agents and for this reason liable to display cutaneous discomfort. In other specific cases, the combination of the disclosure can be applied to the skin when it exhibits clinical signs of deficiency in the cutaneous barrier, for example atopic skin.

Thus, a subject-matter of the disclosure is the use of at least an effective amount of hesperidin or of one of its derivatives in combination with at least an effective amount of at least one microorganism, in particular probiotic microorganism, or one of its fractions in the preparation of a composition, in particular a dermatological composition, intended to prevent a reduction in and/or to reinforce the barrier function of the skin.

Another aspect of the present disclosure is thus the use of at least an effective amount of hesperidin or of one of its derivatives in combination with at least an effective amount of at least one microorganism, in particular probiotic microorganism, or one of its fractions in the preparation of a composition, in particular a dermatological composition, intended to prevent a reduction in and/or to reinforce the barrier function of injured skin, in particular atopic skin.

The combination according to the disclosure or a composition comprising such a combination according to the disclosure can in particular be intended to prolong the phases of remission between acute crises of dermatological conditions, for example of atopy type.

What is more, this prevention of a reduction in and/or this reinforcement of the cutaneous barrier function makes it possible to render it more resistant, in particular to pollutants and to solar radiation, and in fact to protect the living tissues of the skin from the detrimental effects (maximized by UV radiation) of gases, heavy metals and organic compounds which are combustion residues.

The Inventors have thus discovered that the combination under consideration according to the disclosure can advantageously display a protective activity at the level of the cutaneous barrier function and can thus make it possible to limit the penetration of the various pollutants and to increase the resistance of the skin to the attacks.

It has thus also been found that a composition comprising at least an effective amount of hesperidin or of one of its derivatives in combination with an effective amount of at least one microorganism, in particular probiotic microorganism, or one of its fractions makes it possible to preserve and to protect the skin from the harmful effects of pollution.

In addition, the Inventors have also found that the use of the combination under consideration according to the disclosure proves to be particularly effective, in particular in adults, in the treatment of cutaneous signs of ageing and/or photoageing of the skin brought about by a deficiency in the barrier function and in particular induced or exacerbated by pollution, while protecting the barrier function of the skin.

Thus, the present disclosure relates, according to another of its aspects, to a cosmetic and/or dermatological composition, in particular of use in preventing a reduction in and/or reinforcing the barrier function of the skin and especially in preventing and/or treating cutaneous signs of ageing and/or photoageing, especially those induced or exacerbated by pollution, and/or disorders associated with cutaneous dryness comprising, in a physiologically acceptable carrier, at least an effective amount of hesperidin or of one of its derivatives in combination with at least an effective amount of at least one microorganism, in particular probiotic microorganism, or one of its fractions.

The present disclosure also relates to the cosmetic use of at least an effective amount of hesperidin or of one of its derivatives in combination with at least an effective amount of at least one microorganism, in particular probiotic microorganism, or one of its fractions as agent for preventing and/or treating cutaneous signs of ageing and/or photoageing, in particular induced or exacerbated by pollution.

It also relates, according to another of its aspects, to the cosmetic use of at least an effective amount of hesperidin or of one of its derivatives in combination with at least an effective amount of at least one microorganism, in particular probiotic microorganism, or one of its fractions as agent for combating pollution.

The present disclosure also relates, according to another of its aspects, to the use of at least an effective amount of hesperidin or of one of its derivatives in combination with at least an effective amount of at least one microorganism, in particular probiotic microorganism, or one of its fractions in the preparation of a composition, in particular a cosmetic and/or dermatological composition, intended to prevent and/or treat cutaneous signs of ageing and/or photoageing, in particular induced or exacerbated by pollution.

The present disclosure also relates, according to another of its aspects, to a method for the cosmetic treatment of the skin intended to prevent a reduction in and/or to reinforce the barrier function of the skin comprising the administration of at least an effective amount of hesperidin or of one of its derivatives in combination with at least an effective amount of at least one microorganism, in particular probiotic microorganism, or one of its fractions.

The present disclosure also relates, according to another of its aspects, to a cosmetic treatment method for preventing and/or treating disorders associated with dryness of keratinous substances and in particular dry skin, comprising the administration, for example to a subject having a dry skin, of at least an effective amount of hesperidin or of one of its derivatives in combination with at least an effective amount of at least one microorganism, in particular probiotic microorganism, or one of its fractions.

The present disclosure also relates, according to another of its aspects, to a method for the cosmetic treatment of cutaneous signs of ageing and/or photoageing, in particular signs induced or exacerbated by pollution, comprising the administration of at least an effective amount of hesperidin or of one of its derivatives in combination with at least an effective amount of at least one microorganism, in particular probiotic microorganism, or one of its fractions.

A method according to the disclosure can in particular comprise at least one stage which consists in applying, to the skin of individuals exhibiting fragile or delicate skin and/or to the skin of individuals exhibiting weakened skin, in particular the skin of individuals of at least 60 years, indeed even of at least 75 years, and/or to the skin of individuals exhibiting abused skin or an area of abused skin, in particular the shaven skin of the face or body and/or skin suffering from dryness, at least an effective amount of hesperidin or of one of its derivatives in combination with at least an effective amount of at least one microorganism, in particular probiotic microorganism, or one of its fractions.

### DETAILED DESCRIPTION OF EMBODIMENTS

Unless otherwise indicated, in the context of the disclosure, the term “skin” is understood to mean any cutaneous surface of the body including the skin and widened to the scalp and to the mucous and semimucous membranes.

Within the meaning of the present disclosure, the term “to prevent” is understood to mean the fact of reducing the risk of onset of a phenomenon.

Within the meaning of the disclosure, the expression “to prevent a reduction in the barrier function of the skin” means to prevent any detrimental change in the said barrier function below its level of natural effectiveness and which would have the consequence of initiating the appearance of one or more cutaneous disorders as defined above.

Within the meaning of the present disclosure, the expression “keratinous substance” is intended to denote the skin, scalp, mucous and semimucous membranes, nails and keratinous fibres of human or animal origin.

The term “effective amount” is understood to mean, within the meaning of the present disclosure, an amount sufficient to produce the expected effect.

The term “cutaneous signs of ageing and/or photoageing of the skin brought about by a deficiency in the barrier function” is understood to mean, within the meaning of the present disclosure, detrimental changes in the dermal and/or epidermal tissue, dull or nonsupple skin, slackened by a detrimental change in the elastic fibres, a flaccid appearance of the skin, a loss in tonicity, a detrimental change in the microrelief of the skin or in the oval of the face, dyschromia, and the like.

The combination according to the disclosure can be formulated in cosmetic or dermatological compositions.

According to one embodiment, the use or the method according to the disclosure can comprise the application of the combination according to the disclosure via the topical route, or oral or parenteral administration.

The term “topical route” is understood to mean the administration of the combination according to the disclosure or of the compositions which comprise it by application to the skin as defined above.

According to another embodiment, the use or the method according to the disclosure can comprise the administration of the combination according to the disclosure via the aerial or subcutaneous route.

The subcutaneous administration can in particular be carried out using a syringe.

As indicated above, the topical and oral routes can be envisaged for the implementation of the disclosure.

Nevertheless, by definition, topical products act locally on the areas to be treated, over which areas they may be unevenly distributed, and require careful and repeated applications. In addition they may in some cases be the cause of cutaneous side reactions, indeed even of discomfort.

In contrast, the oral route exhibits the advantage of comprehensively influencing the whole of the skin and in its deep layers (dermis, hypodermis), according to a fast and not very restrictive method of administration. Specifically, the metabolites and other active nutriments are in particular distributed in the dermal matrix via the blood circulation. The oral route or the administration via a patch also exhibits the advantage of a fast and not very restrictive method of administration.

According to a preferred embodiment, the cosmetic use according to the disclosure is thus carried out via the oral route and the method according to the disclosure comprises the administration via the oral route of the said combination according to the disclosure.

### Microorganisms and in Particular Probiotic Microorganisms

The microorganisms suitable for the disclosure are microorganisms which can be administered without risks to animals or humans.

In particular, use is made, in the present disclosure, of at least one microorganism said to be of probiotic type.

Within the meaning of the present disclosure, the term “probiotic microorganism” is understood to mean a living microorganism which, when it is consumed in an appropriate amount, has a positive effect on the health of its host (“Joint FAO/WHO Expert Consultation on Evaluation of Health and Nutritional Properties of Probiotic in Food Including Powder Milk with Live Lactic Acid Bacteria, 6 Oct. 2001”) and which can in particular improve the intestinal microbial balance.

According to an alternative form of the disclosure, this microorganism is employed in an isolated form, that is to say a form not mixed with one or more of the compound(s) capable of being combined with it in its medium of origin.

Within the meaning of the disclosure, the term “fraction” particularly denotes a fragment of the said microorganism which is effective in the treatment of dry skin by analogy with the said whole microorganism.

The microorganisms suitable for the disclosure can in particular be chosen from Ascomycetes, such as Saccharomyces, Yarrowia, Kluyveromyces, Torulaspora, Schizosaccharomyces pombe, Debaromyces, Candida, Pichia, Aspergillus and Penicillium, bacteria of the genus Bifidobacterium, Bacteroides, Fusobacterium, Melissococcus, Propionibacteriunm, Enterococcus, Lactococcus, Staphylococcus, Peptostrepococcus, Bacillus, Pediococcus, Micrococcus, Leuconostoc, Weissella, Aerococcus, Oenococcus or Lactobacillus, and their mixtures.

Mention may in particular be made, as Ascomycetes very particularly suitable for the present disclosure, of Yarrowia lipolitica and Kluyveromyces lactis, and also Saccharomyces cereviseae, Torulaspora, Schizosaccharamyces pombe, Candida and Pichia.

As regards the probiotic microorganisms, it is the following bacterial and yeast genera which are generally used:
lactic bacteria: which produce sugar by fermentation of lactic acid. According to their morphology, they are divided into two groups:
   Lactobacillus species: Lactobacillus acidophilus; amylovorus, casei, rhamnosus, brevis, crispatus, delbrueckii (subsp bulgaricus, lactis), fermentum, helveticus, gallinarum, gasseri, johnsonii, paracasei, plantarum, reuteri, salivarius, alimentarius, curvatus, casei subsp. casei, sake
   Gocci: Enterococcus (faecalis, faecium), Lactococcus lactis (subsp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus carnosus, Staphylococcus xylosus
bifidobacteria or Bifidobacterium species: Bifidobacterium adolescentis, animalis, bifidum, breve, lactis, longum, infantis, pseudocatenulatum,
yeasts: Saccharomyces (cerevisiae or also boulardii),
other spore-forming bacteria: Bacillus (cereus var toyo or subtilis), Bacillus coagulans, Bacillus licheniformis, Escherichia coli strain nissle, Propionibacterium freudenreichii,
and their mixtures.

The lactic bacteria and the bifidobacteria are the probiotics most often used.

Specific examples of probiotic microorganisms are Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (NCFB 1748); Lactobacillus amylovorus, Lactobacillus casei (Shirota), Lactobacillus rhamnosus (strain GG), Lactobacillus brevis, Lactobacillus crispatus, Lactobacillus delbrueckii (subsp bulgaricus, lactis), Lactobacillus fermentum, Lactobacillus helveticus, Lactobacillus gallinarum, Lactobacillus gasseri, Lactobacillus johnsonii (CNCM I-1225), Lactobacillus paracasei, Lactobacillus plantarum, Lactobacillus reuteri, Lactobacillus salivarius, Lactobacillus alimentarius, Lactobacillus curvatus, Lactobacillus casei subsp. casei, Lactobacillus sake, Lactococcus lactis, Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus carnosus, Staphylococcus xylosus, Saccharomyces (cerevisiae or also boulardii), Bacillus (cereus var toyo or subtilis), Bacillus coagulans, Bacillus licheniformis, Escherichia coli strain nissle, Propionibacterium freudenreichii and their mixtures.

Preferably, the probiotic microorganism can be chosen from: Lactobacillus acidophilus, Lactobacillus alimentarius, Lactobacillus curvatus, Lactobacillus delbruckii (subsp bulgaricus lactis), Lactobacillus gasseri, Lactobacillus johnsonii, Lactobacillus reuteri, Lactobacillus rhamnosus (strain GG), Lactobacillus sake, Lactococcus lactis, Streptococcus thermophilus, Staphylococcus carnosus and Staphylococcus xylosus and their mixtures.

These microorganisms can be formulated in the form of powders, that is to say in a dry form, or in the form of suspensions or solutions.

More particularly, they are probiotic microorganisms resulting from the group of the lactic bacteria, such as in particular the Lactobacillus species and/or the Bifidobacterium species. Mention may more particularly be made, by way of illustration of these lactic bacteria, of Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum and their mixtures and preferably Lactobacillus johnsonii, Lactobacillus reuteri, Lactobacillus rhamnosus, Lactobacillus paracasei, and their mixtures.

The species which are very particularly suitable are Lactobacillus johnsonii, Lactobacillus paracasei, Bifidobacterium adolescentis, Bifidobacterium longum and Bifidobacterium lactis NCC 2818, respectively deposited according to the Treaty of Budapest with the Institut Pasteur (28 rue du Docteur Roux, F-75024 Paris cedex 15) on Jun. 30, 1992, Jan. 12, 1999, Apr. 15, 1999, Apr. 15, 1999 and Jul. 6, 2005 under the following designations CNCM I-1225, CNCM I-2116, CNCM I-2168, CNCM I-2170 and CNCM I-3446, and the genus Bifidobacterium longum (BB536) and their mixtures.

According to a specific embodiment, the combination employs, with hesperidin or with one of its derivatives, at least one probiotic microorganism of the genus Lactobacillus species, and more particularly of the species Lactobacillus paracasei, or one of its fractions.

According to one embodiment, the probiotic microorganism is the strain Lactobacillus paracasei deposited according to the Treaty of Budapest with the Institut Pasteur (28 rue du Docteur Roux, F-75024 Paris cedex 15) on Dec. 1, 1999 under the designation CNCM I-2116.

According to a specific embodiment, the combination according to the disclosure can comprise at least two different microorganisms, in particular probiotic microorganisms, and/or fractions of these.

The microorganism(s) can be included in the composition according to the disclosure in a living, semi-active or inactivated, or dead form.

It/they can also be included in the form of fractions of cellular components. The microorganism(s) or fraction(s) can also be introduced in the form of a lyophilized powder or of a culture supernatant and/or, if appropriate, in a concentrated form.

Generally, the compositions according to the disclosure can comprise from 0.00001 to 20% by weight, in particular from 0.001 to 20% by weight and more particularly from 0.01 to 10% by weight of microorganism(s), in particular probiotic microorganism(s), with respect to the total weight of the composition.

It can be advantageous to employ these microorganisms in the inactivated, indeed even dead, form and more particularly in the form of a lysate.

Such a lysate can be obtained from the cell lysis of the microorganism concerned, according to a conventional method.

The probiotic microorganism(s) in the form of a disintegrated lysate in suspension can be formulated in an appropriate carrier in a proportion of less than 20% by weight, in particular in a proportion of 0.0001 to 20% by weight and more particularly in a proportion of 0.01 to 10% by weight, with respect to the total weight of the said carrier.

When they are living, the microorganisms and/or their fractions can be formulated in an appropriate carrier in an amount equivalent to at least 10³ ufc/g, in particular to doses varying from 10⁵ to 10¹⁵ ufc/g and more particularly from 10⁷ to 10¹² ufc/g of carrier.

Consequently, the compositions according to the disclosure generally comprise from 10³ to 10¹² ufc, in particular from 10⁵ to 10¹⁰ ufc and more particularly from 10⁷ to 10⁹ ufc of living microorganisms, in particular probiotic microorganisms, per gram of carrier.

Hesperidin

Hesperidin belongs to the family of the flavanones, which are natural glucoside compounds found mainly in citrus fruits, that is to say fruits of the genus Citrus, such as, for example, oranges, lemons or bitter oranges, or also grapes.

They are present predominantly in the peel of citrus fruits but are also found in large amounts in the pulp and thus in the juice of citrus fruits.

Hesperidin is a glucosylated compound comprising a flavanone nucleus of hesperitin (3′,5,7-trihydroxy-4′-methoxyflavanone) to which is covalently bonded a glycoside part formed of rutinose L-rhamnosyl-(α1→6)-glucose) attached to the hydroxyl group present on the carbon in the 7 position of the hesperitin.

“Hesperidin” is thus intended to mean the compound (S)-7-[[6-O-(6-deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one.

The hesperidin derivatives can be chosen from its aglycone forms, its chalcone forms, its glycosylated forms and its methylated forms, and also its sulphate or glucuronide forms which are found as metabolic products in the blood circulation.

Hesperidin derivatives can be obtained by various processes known to a person skilled in the art, such as, for example, enzymatic treatments, or can also be obtained by synthesis. Glucose-7-hesperitin can thus be prepared by treatment with rhamnosidase or hesperidinase.

Mention may in particular be made, as hesperidin derivative, of the following compounds:
the compound hesperitin, composed of the nonglycosylated flavanone nucleus of hesperidin, which has the following formula: (S)-2,3-dihydro-5,7-dihydroxy-2-(3-hydroxy-4-methoxyphenyl)-4H-1-benzopyran-4-one; 3′,5,7-trihydroxy-4′-methoxy-flavanone;
α-glucosyl hesperidin, which comprises a chain of 1 to 20 glucose residues bonded to one another via a 1,4 bond, the chain of glucose residues being itself bonded via a bond of 1,4 type in the 4 position of the glucose residue of the hesperidin; these hesperidin derivatives and their process of preparation are described in particular in Patent Application EP 0 825 196 and patent U.S. Pat. No. 6,048,712;
methyl hesperidin compounds, in particular the compound 3′-methyl-7-(rhamnosyl-2-methylglucosyl)hesperitin and the compound 3′-methylhesperitin, these compounds, and their process of preparation, being described in U.S. Pat. No. 858,784;
sulphate or glucuronide conjugates of hesperitin, which are found, with hesperitin, as products of the metabolisation of hesperidin in the blood circulation.

According to one embodiment, hesperidin and its derivatives can be chosen from hesperitin and hesperitin glucuronide.

Generally, the effective amount of hesperidin or of one of its derivatives can be employed in a proportion of 0.00001 to 20% by weight, for example from 0.001 to 10% by weight or else from 5% to 10% by weight, with respect to the total weight of a composition comprising it.

Generally, in the process and uses according to the disclosure, the dose of hesperidin or one of its derivatives is employed so that to administer, namely by oral route, between 100 mg and 1000 mg, preferably between 200 mg and 800 mg, preferably between 300 mg and 600 mg and about 500 mg of hesperidin or one of its derivatives per person and per day.

Advantageously, a composition according to the present disclosure, namely intended for administration by oral route, comprises an amount of hesperidin or one of its derivatives comprised between 100 mg and 800 mg, preferably between 200 mg and 800 mg, preferably between 300 mg and 600 mg and about 500 mg.

Such a composition may namely be under the form of a capsule.

The compositions according to the disclosure can be provided in all the formulation forms normally available for the method of administration selected.

The carrier can be of various natures according to the type of composition under consideration.

As regards more particularly the compositions intended for administration by the external topical route, that is to say on the skin, they can be aqueous, aqueous/alcohol or oily solutions, dispersions of the type of solutions or dispersions of the lotion or serum type, emulsions with a liquid or semiliquid consistency of the milk type, suspensions or emulsions of the cream type, aqueous or anhydrous gels, microemulsions, microcapsules, microparticles or vesicular dispersions of ionic and/or nonionic type.

These compositions are prepared according to the usual methods.

In a known way, the formulation forms intended for topical administration can also comprise adjuvants usual in the cosmetic, pharmaceutical and/or dermatological field, such as hydrophilic or lipophilic gelling agents, hydrophilic or lipophilic active principles, preservatives, antioxidants, solvents, fragrances, fillers, screening agents, bactericides, odour absorbers and colouring materials. The amounts of these various adjuvants are those conventionally used in the field under consideration, for example from 0.01 to 20% of the total weight of the composition. These adjuvants, according to their nature, can be introduced into the fatty phase and/or into the aqueous phase.

Mention may be made, as fatty substances which can be used in the disclosure, of mineral oils, such as, for example, hydrogenated polyisobutene and liquid petrolatum, vegetable oils, such as, for example, a liquid fraction of shea butter, sunflower oil and apricot kernel oil, animal oils, such as, for example, perhydrosqualene, synthetic oils, in particular Purcellin oil, isopropyl myristate and ethylhexyl palmitate, unsaturated fatty acids and fluorinated oils, such as, for example, perfluoropolyethers. Use may also be made of fatty alcohols, fatty acids, such as, for example, stearic acid, and such as, for example, waxes, in particular paraffin wax, carnauba wax and beeswax. Use may also be made of silicone compounds, such as silicone oils, for example cyclomethicones and dimethicones, silicone waxes, silicone resins and silicone gums.

Mention may be made, as emulsifiers which can be used in the disclosure, for example, of glycerol stearate, polysorbate 60, the cetearyl alcohol/oxyethylenated cetearyl alcohol comprising 33 mol of ethylene oxide mixture sold under the name Sinnowax AO® by Henkel, the PEG-6/PEG-32/Glycol Stearate mixture sold under the name Tefose® 63 by Gattefossé, PPG-3 myristyl ether, silicone emulsifiers, such as cetyl dimethicone copolyol, and sorbitan mono- or tristearate, PEG-40 stearate, oxyethylenated (20 EO) sorbitan monostearate.

Mention may be made, as solvents which can be used in the disclosure, of lower alcohols, in particular ethanol and isopropanol, or propylene glycol.

The composition of the disclosure can also advantageously comprise a thermal and/or mineral water, in particular chosen from water from Vittel, water from the Vichy basin and water from La Roche-Posay.

In the case of the use in accordance with the disclosure by the oral route, the use of an ingestible carrier is favoured.

The ingestible carrier can be of various natures according to the type of composition under consideration.

In particular, tablets, including compressed ones, oral supplements in the dry form and oral supplements in the liquid form are thus suitable as food or pharmaceutical carriers.

They can, for example, be food supplements, the formulation of which can be carried out by standard processes for producing in particular tablets, including sugar-coated tablets, capsules, including hard gelatin capsules, gels, emulsions and hydrogels which make possible controlled release.

In particular, the combination according to the disclosure can be incorporated in all other forms of food supplements or enriched foods, for example food bars or compacted or uncompacted powders. The powder can be diluted in water, fizzy drinks, dairy products or soya derivatives or can be incorporated in food bars.

The combination according to the disclosure can furthermore be formulated with excipients and components conventional for such oral compositions or food supplements, namely in particular fatty and/or aqueous components, humectants, thickeners, preservatives, texturizing, flavouring and/or coating agents, antioxidants and colorants normal in the field of food.

The formulating agents and excipients for oral compositions and in particular for food supplements are known in this field and do not form here the subject of a detailed description.

Milk, yoghourt, cheese, fermented milks, milk-based fermented products, ice creams, cereal-based products or products based on fermented cereals, milk-based powders, formulations for children and infants, foodstuffs of confectionery, chocolate or cereal type, foods for animals, in particular domestic animals, tablets, including compressed tablets, hard gelatin capsules, liquid bacteria suspensions, oral supplements in the dry form and oral supplements in the liquid form are suitable in particular as dietary or pharmaceutical carriers.

Whatever the method of administration under consideration, the combination according to the disclosure can also advantageously be combined with at least one other active principle.

Mention may be made, as active principles which can be used, of vitamins A, B3, B5, B6, B8, C, D, F or PP, curcuminoids, carotenoids, polyphenol compounds and minerals, sugars, amino acids, sulphur-comprising amino acids, 3 and 6 polyunsaturated fatty acids, taurine and phytosterols.

Use may in particular be made of an antioxidant complex comprising vitamins C and E and at least one carotenoid, in particular a carotenoid chosen from β-carotene, lycopene, astaxanthin, zeaxanthin and lutein, flavonoids, such as catechins, proanthocyanidins, anthocyanins, ubiquinones, coffee extracts comprising polyphenols and/or diterpenes, chicory extracts, ginkgo biloba extracts, grape extracts rich in proanthocyanidins, pepper extracts, soybean extracts, other sources of flavonoids having antioxidant properties, fatty acids, prebiotics, taurine, resveratrol, selenium amino acids or glutathione precursors.

Among the flavonoids, catechins and OPCs (procynidol oligomers) are preferably chosen.

Use may more particularly be made, in topical formulation forms, as hydrophilic active principles, of proteins or protein hydrolysates, amino acids, polyols, in particular C₂ to C₁₀ polyols, such as glycerol, sorbitol, butylene glycol and polyethylene glycol, urea, allantoin, sugars and sugar derivatives, water-soluble vitamins, starch, or bacterial or plant extracts, such as those of aloe vera.

As regards the lipophilic active principles, use may be made of retinol (vitamin A) and its derivatives, tocopherol (vitamin E) and its derivatives, ceramides, essential oils and nonsaponifiable materials (tocotrienol, sesamin, γ-oryzanol, phytosterols, squalenes, waxes or terpenes).

Consideration may also be given, as active principles also capable of being combined with the combination according to the disclosure, suitable for the topical route but more particularly suitable for an oral formulation formula, to all the ingredients commonly used and/or authorized, in particular active agents intended to prevent and/or treat skin complaints.

Mention may be made, by way of illustration, of vitamins, minerals, essential lipids, trace elements, polyphenols, flavonoids, phyto-oestrogens, antioxidants, such as lipoic acid and coenzyme Q10, carotenoids, prebiotics, proteins and amino acids, mono- and polysaccharides, amino sugars, phytosterols and triterpene alcohols of plant origin.

They are in particular vitamins A, C, D, E, PP and of the group B. The choice is preferably made, among carotenoids, of β-carotene, lycopene, lutein, zeazanthin and astaxanthin. The minerals and trace elements particularly employed are zinc, calcium, magnesium, copper, iron, iodine, manganese, selenium or chromium(III). The selection is also in particular made, among polyphenol compounds, of grape, tea, olive, cocoa, coffee, apple, blueberry, elder, strawberry, cranberry and onion polyphenols. The selection is preferably made, among phyto-oestrogens, of isoflavones in the free or glycosylated form, such as genistein, daidzein or glycitein, or also lignans, in particular those of flax and of Schisandra chinensis. Amino acids or peptides and proteins comprising them, such as taurine, threonine, cysteine, tryptophan or methionine. The lipids preferably belong to the group of the oils comprising mono- and polyunsaturated fatty acids, such as oleic, linoleic, α-linolenic, γ-linolenic or stearidonic acids, long-chain fish ω-3 fatty acids, such as EPA and DHA, or conjugated fatty acids originating from plants or animals, such as CLAs (Conjugated Linoleic Acid).

Thus, in particular when the combination according to the disclosure is intended for administration by the oral route, it can additionally be combined with at least one nutritional active principle chosen from lycopene, vitamin C, vitamin E and polyphenol compounds.

The combination according to the disclosure can also be combined with other nutritional active principles chosen from:

anti-ageing nutritional active principles, such as dietary antioxidants, nutriments having properties in combating free radicals and cofactors of endogenous antioxidant enzymes, vitamins A, C or E, carotenoids, xanthophylls, isoflavones, some minerals, such as zinc, copper, magnesium or selenium, lipoic acid, coenzyme Q10, superoxide dismutase (SOD) or taurine. Mention may in particular be made, among anti-ageing active principles, of nonsaponifiable fractions extracted from lipids of plant origin, aloe vera, native or hydrolysed marine collagen, or vegetable or marine oils rich in ω-3 or ω-6 fatty acids (including γ-linolenic acid),

photoprotective nutritional active principles, such as: antioxidants and agents for combating free radicals: vitamins A, C or F, carotenoids, xanthophylls, some minerals, such as zinc, copper, magnesium or selenium, coenzyme Q10 or superoxide dismutase (SOD),

nutritional ingredients exhibiting moisturizing or immunomodulating properties, such as Polypodium leucotomos extract, vegetable or marine oils rich in ω-3 or ω-6 fatty acids, including γ-linolenic acid,

nutritional active principles which are active with regard to clinical signs of the menopause (for example hot flushes, and the like), such as isoflavones, lignans, DHEA, yam, sage or hop extracts, calcium, magnesium, protein hydrolysates, or vegetable or marine oils rich in ω-3 fatty acids,

nutritional ingredients employed in the field of slimming, such as extracts of green tea, maté, horse chestnut, kola, caffeine, theobromine, synephrine, bromelain, Ephedra, Citrus aurantium, calcium, Hoodia, Garcinia, chitosan, plant fibres (cactus, apples, pineapple, and the like), fennel, blackcurrant, meadowsweet or black radish.

The disclosure also relates to a cosmetic treatment method for preventing a reduction in and/or reinforcing the barrier function of the skin, in particular for the care of elderly skin, comprising at least one stage of administration of the combination according to the disclosure.

The cosmetic treatment method of the disclosure can be employed in particular by administering, by the oral and/or topical route, at least one combination according to the disclosure.

The administration by the topical route consists of the application of cosmetic and/or dermatological compositions or of combinations as defined above according to the usual technique for the use of these compositions.

The cosmetic method according to the disclosure can be carried out by topical administration, for example daily, of cosmetic and/or dermatological compositions or of the combination according to the disclosure, which can, for example, be formulated in the form of gels, lotions or emulsions.

The administration by the oral route consists in ingesting, all at once or at intervals, an oral composition as defined above.

According to an alternative form, the cosmetic method comprises at least one stage of oral administration of the combination according to the disclosure and at least one stage of topical administration of the combination according to the disclosure.

The method according to the disclosure can comprise a single administration.

According to another embodiment, the administration is repeated, for example, 2 to 3 times daily over one day or more and generally over a prolonged period of at least 4 weeks, indeed even 4 to 15 weeks, with, if appropriate, one or more periods of interruption.

In the description and in the following examples, unless otherwise indicated, the percentages are percentages by weight and the ranges of values worded in the form “between . . . and . . . ” include the lower and upper limits specified.

The ingredients are mixed, before their forming, in the order and under conditions easily determined by a person skilled in the art.

The following examples and FIGURE are presented by way of illustration and without implied limitation of the field of the disclosure.

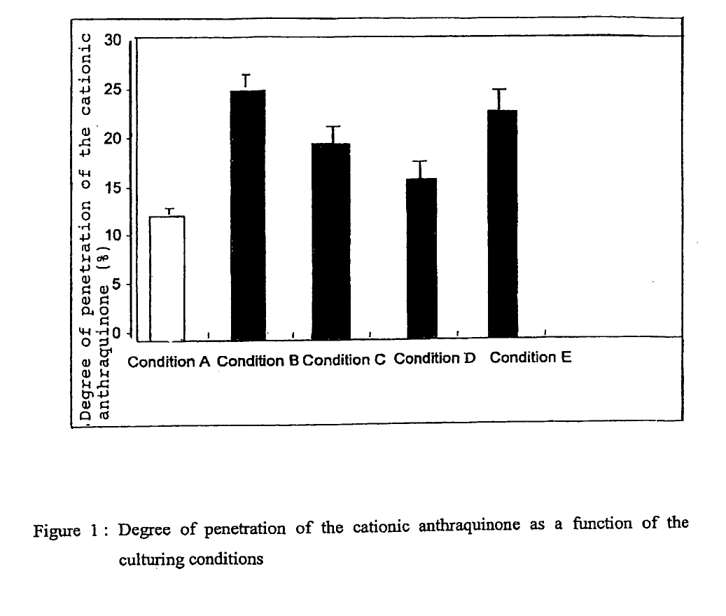

FIG. 1: Degree of penetration of the cationic anthraquinone as a function of the culturing conditions (represented by the letters A, B, C, D and E).

### EXAMPLE 1

### Evaluation of the Effect of a Combination According to the Invention on the Barrier Function of Reconstituted Skin

The conversion of a nutritional agent (hereinafter known as probiotic conditioned medium) after ingestion is first of all stimulated, for the purpose of resulting use directly on a skin model (stage A).

The medium of the cocultures stimulated with Lactobacillus paracasei, resulting from the basolateral compartment, is subsequently withdrawn and its effects, in combination with the most important metabolite of hesperidin, hesperitin-7 glucuronide (Hes7Glu), on the barrier function in the Episkin® model are subsequently tested in vitro (stage B).

A—Preparation of the Probiotic Conditioned Medium

An intestinal barrier model (comprising human enterocyte lines (Caco-2) co-cultured with human leukocytes in a “transwell” cell co-culture system [Haller D, 2000]) has been developed.

This model consists in separately culturing:
an intestinal cell line Caco-2 on “transwell” inserts, which are subsequently placed in a 12-well plate (Nunc), where the cells are cultured for 21 days; and
human peripheral blood mononuclear cells (leukocytes), which are purified and then resuspended in an appropriate culture medium.

This suspension of leukocytes is then added to the basolateral compartment of the “transwell” cultures when the latter exhibit a confluent layer of Caco-2 cells.

The cocultures thus established are stimulated by adding 1×10⁷ CFU/ml of probiotic microorganism at the apical surface of the monolayer of epithelial cells (Caco-2). The system is subsequently incubated for 16 h at 37° C./5% CO₂.

At the end of incubation (16 h), the medium found in the basolateral compartment is withdrawn in order to be tested.

This model of coculturing intestinal cells and leukocyte cells makes it possible to simulate the cell interactions present during the injection by the oral route of a nutritional ingredient and to mimic in vitro the situation in vivo.

The interaction of the activated or nonactivated enterocytes with nutritional agents, such as probiotics, which acts at the apical level, results in stimulation of the underlying leukocytes and the production of mediators (cytokines). Under the effect of stimulation by probiotic microorganisms, these mediators or other immunoregulatory molecules produced in the intestinal mucous membrane are carried by the blood to the skin, where they contribute to its reinforcement and/or to counterbalancing a local inflammatory reaction.

B—Measurement of the Effect of Several Nutritional Agents on a Reconstituted Skin Model

The Episkin® kits were received on D6 and then cultured during the proliferative phase up to day 13 according to five conditions:

1. Conventional Episkin Condition (Condition A)

Treated from D6 to D13 with the Episkin® differentiation medium

2. Negative Control (Condition B)

Treated from D6 to D13 with 30% of negative control medium (conditioned medium resulting from 16 h 00 of Caco-2/PBMC culturing)

3. Positive Control (Condition C)

Treated from D6 to D13 with 20% of probiotic conditioned medium

4. Positive Control (Condition D)

Treated from D6 to D13 with 20% of probiotic conditioned medium+10 μM Hes7Glu

5. Positive Control (Condition E)

Treated from D6 to D13 with 10 μM Hes7Glu

Conditions A, B, C, D and E were studied on 6 wells for each Episkin® batch, by measuring the penetration of a nonpenetrating reference compound (cationic anthraquinone) formulated in a simplex medium. The degree of penetration of the cationic anthraquinone makes it possible to characterize the influence of the various culturing conditions on the barrier function of the reconstituted tissue.

Before the application, the culture medium is removed and replaced with 1.5 ml of Episkin test medium, and the kits are placed for 30 minutes in an oven at 37° C., 5% CO₂. A 2nd time, the medium is removed and replaced with fresh medium, and the kits are again placed in an oven for 30 minutes. Finally, the Episkin test medium is replaced with 1.5 ml of PBS+0.25% Tween (w/w) and the kits are placed in a chamber thermostatically controlled at 32° C. with stirring (Certomat).

The nonpenetrating reference dye (cationic anthraquinone) was used. 250 μl of a simplex formulation buffered at pH 7 at a concentration of 1 mM are applied for 4 hours.

The receiving liquid (RL) is collected and then assayed directly at the end of application by HPLC. Each point is analysed in duplicate.

During the development of the analytical method, the specificity was confirmed from RL blanks (receiving liquids obtained after application under the same formulation conditions without dye).

The concentration is determined using a calibration range produced on the same day. The degrees of penetration are calculated from the ratio of the amount in the RL to the amount applied, the following results having in addition formed the subject of a statistical study using the Wilcoxon tests.

Condition A uses the Episkin® differentiation medium as culture medium, which is optimal for the differentiation of the model.

Condition B uses a medium in which 30% of nonstimulated conditioned medium has been incorporated (similar to a conventional Episkin® differentiation culture medium reduced by 30%). Consequently, the barrier function presented by condition B has thus been found to be weaker than that of condition A.

FIG. 1 indicates the degrees of penetration of the cationic anthraquinone reference compound into the receiving liquid for each condition studied.

It is found that the conditioned media resulting from stimulation with 20% Lactobacillus paracasei give results which are significantly different from those obtained under negative control condition B using the nonstimulated medium.

While Hes7Glu, introduced at a concentration of 10 μM, has no significant effect on the barrier function, this same concentration, added at 20% of probiotic conditioned medium, makes it possible to encounter an effectiveness of the barrier function which is as good as that obtained with the conventional Episkin® differentiation medium.

Consequently, these results clearly show a synergy in activity for hesperitin-7-glucuronide in association with the conditioned medium stimulated with the probiotic Lactobacillus paracasei.

The introduction of 10 M of hesperitin-7-glucuronide into the culture medium supplemented with 20% of conditioned media stimulated with the probiotic Lactobacillus paracasei makes it possible to encounter an effective barrier function comparable to that obtained under the standard Episkin® reference conditions.

### EXAMPLE 2

### Single-Dose Gel

| | % by weight |
|---|---|
| Active principle | |
| Hesperidin OBC, sold by Nutrafur (hesperidin in | 10 |
| the 93% pure micronized form) | |
| Lycopene | 10 |
| Lactobacillus johnsonii (CNCM I-1225) | 10¹⁰ cfu |
| Excipient | |
| Sugar syrup | 50 |
| Maltodextrin | 17 |
| Xanthan gum | 0.8 |
| Sodium benzoate | 0.2 |
| Water | q.s. for 100 |

A dose of 200 to 400 ml per day can be taken.

### EXAMPLE 3

### Capsule

| | mg/capsule |
|---|---|
| Hesperidin, sold by Selectchemie (hesperidin | 10 |
| in the 93% pure micronized form) | |
| Lactobacillus paracasei (CNCM I-2116) | 10¹⁰ cfu |
| Glycerol | 150 |
| Magnesium stearate | 0.02 |
| Natural flavouring | q.s. for 100 |

One to three of these capsules per day can be taken.

### EXAMPLE 4

A vitamin complex comprising 60 mg of vitamin C, 100 μg of vitamin E and 6 mg of β-carotene is added to the formulation of Example 2.

### EXAMPLE 5

A vitamin complex comprising 100 mg of vitamin C, 100 μg of vitamin E and 6 mg of lycopene per capsule is added to the formulation of Example 2.

### EXAMPLE 6

### Single-Dose Gel

| | % by weight |
|---|---|
| Active principle | |
| Hesperidin, sold by Selectchemie | 10 |
| (93% pure micronized hesperidin) | |
| Lycopene | 10 |
| Lactobacillus paracasei (CNCM I-2116) | 10¹⁰ cfu |
| Excipient | |
| Sugar syrup | 50 |
| Maltodextrin | 17 |
| Xanthan gum | 0.8 |
| Sodium benzoate | 0.2 |
| Water | q.s. for 100 |

A dose of 200 to 400 ml per day can be taken.

### EXAMPLE 7

### Capsule

| | mg/capsule |
|---|---|
| Vitamin C | 60 |
| Hesperidin OBC, sold by Nutrafur (93% pure | 8 |
| micronized hesperidin) | |
| Lactobacillus paracasei (CNCM I-2116) | 10¹⁰ cfu |
| Glycerol | 150 |
| Magnesium stearate | 0.02 |
| Natural flavouring | q.s. for 100 |

One to three of these capsules per day can be taken.

### EXAMPLE 8

A vitamin complex comprising 60 mg of vitamin C, 100 μg of vitamin E and 6 mg of β-carotene is added to the formulation of Example 7.

### EXAMPLE 9

A vitamin complex comprising 100 mg of vitamin C, 100 μg of vitamin E and 6 mg of lycopene per capsule is added to the formulation of Example 7.

EXAMPLE 10
Cream for the care of the face

| | (% by weight) |
|---|---|
| Hesperitin | 5.00 |
| Lactobacillus paracasei (CNCM I-2116) | 10.00 |
| Antioxidant | 0.05 |
| Isopropanol | 40.00 |
| Glyceryl stearate | 1.00 |
| Cetearyl alcohol/oxyethylenated cetearyl alcohol | 3.00 |
| comprising 33 mol of EO | |
| (Sinnowax AO, sold by Henkel) | |
| Cetyl alcohol | 1.00 |
| Dimethicone (DC 200 Fluid, sold by Dow Corning) | 1.00 |
| Liquid petrolatum | 6.00 |
| Isopropyl myristate (Estol IPM 1514, sold by Unichema) | 3.00 |
| Antioxidant | 0.05 |
| Glycerol | 20.00 |
| Preservative | 0.30 |
| Water | q.s. for 100.00 |

EXAMPLE 11
Lotion for the care of the body

| | (% by weight) |
|---|---|
| Hesperitin glucuronide | 5.00 |
| Lactobacillus paracasei (CNCM I-2116) | 10.00 |
| Antioxidant | 0.05 |
| Isopropanol | 40.00 |
| Preservative | 0.30 |
| Water | q.s. for 100.00 |

EXAMPLE 12
Lotion for the hands

| | (% by weight) |
|---|---|
| Hesperitin | 5.00 |
| Lactobacillus paracasei (CNCM I-2116) | 10.00 |
| Antioxidant | 0.05 |
| Isopropanol | 40.00 |
| Preservative | 0.35 |
| Water | q.s. for 100.00 |

EXAMPLE 13
Gel for the care of the body

| | (% by weight) |
|---|---|
| Hesperitin glucuronide | 5.00 |
| Lactobacillus paracasei (CNCM I-2116) | 10.00 |
| Antioxidant | 0.05 |
| Vitamin C | 2.50 |
| Antioxidant | 0.05 |
| Isopropanol | 40.00 |
| Preservative | 0.30 |
| Water | q.s. for 100.00 |

### EXAMPLE 14

### Capsule

| | mg/capsule |
|---|---|
| Hesperidin, sold by Selectchemie (hesperidin | 500 |
| in the 93% pure micronized form) | |
| Lactobacillus paracasei (CNCM I-2116) | 10⁹ cfu |
| Glycerol | 150 |
| Magnesium stearate | 0.02 |
| Natural flavouring | q.s. for 1000 |

One the three of these capsules per day can be taken.

### EXAMPLE 15

### Capsule

| | mg/capsule |
|---|---|
| Vitamin C | 60 |
| Hesperidin OBC, sold by Nutrafur (93% | 500 |
| pure micronized hesperidin) | |
| Lactobacillus paracasei (CNCM I-2116) | 10⁹ cfu |
| Glycerol | 150 |
| Magnesium stearate | 0.02 |
| Natural flavouring | q.s. for 1000 |

One to three of these capsules per day can be taken.

Although the present disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A cosmetic method for preventing a reduction in and/or reinforcing the barrier function of the skin comprising administering to a subject at least an effective amount of hesperidin or of one of its derivatives in combination with at least an effective amount of a least one microorganism or one of its fractions as agent.

2. A cosmetic method for reinforcing the protection of the skin with regard to external attacks comprising administering to a subject at least an effective amount of hesperidin or of one of its derivatives in combination with at least an effective amount of at least one microorganism or one of its fractions as agent.

3. The method according to claim 1, in which the combination is intended to prevent and/or reduce cutaneous discomfort of the skin induced in particular by an exogenous stress of chemical, environmental or mechanical origin and/or an endogenous stress.

4. The method according to claim 3, in which the cutaneous discomfort is characterized by tightness, smarting, a feeling of tautness, inflammation and/or itching.

5. The method according to claim 1, in which the combination is intended to prevent a reduction in and/or to reinforce the barrier function of skin selected from the group consisting of fragile skin especially atopic skin, weakened skin, abused skin, and skin suffering from dryness.

6. A cosmetic method for preventing and/or treating dry keratinous substances and associated disorders thereof comprising administering to a subject an effective amount of hesperidin or of one of its derivatives in combination with at least an effective amount of at least one microorganism or of one of its fractions as agent.

7. A method of making a composition intended to prevent and/or treat dry keratinous substances and associated disorders thereof comprising combining an effective amount of hesperidin or of one of its derivatives in combination with at least an effective amount of at least one microorganism or of one of its fractions with a physiologically acceptable carrier.

8. A cosmetic method for preventing and/or treating cutaneous signs of ageing and/or photoageing comprising administering to a subject at least an effective amount of hesperidin or of one of its derivatives in combination with at least an effective amount of at least one microorganism or one of its fractions as agent.

9. A cosmetic method for combating pollution comprising administering to a subject at least an effective amount of hesperidin or of one its derivatives in combination with at least an effective amount of at least one microorganism or one of its fractions as agent.

10. The method according to claim 1, in which the microorganism is selected from the group consisting of Ascomycetes, bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus, and their mixtures.

11. The method according to claim 1, in which the microorganism is selected from the group consisting of Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (NCFB 1748); Lactobacillus amylovorus, Lactobacillus casei (Shirota), Lactobacillus rhamnosus (strain GG), Lactobacillus brevis, Lactobacillus crispatus, Lactobacillus delbrueckii (subsp bulgaricus, lactis), Lactobacillus fermentum, Lactobacillus helveticus, Lactobacillus gallinarum, Lactobacillus gasseri, Lactobacillus johnsonii (CNCM I-1225), Lactobacillus paracasei, Lactobacillus plantarum, Lactobacillus reuteri, Lactobacillus salivarius, Lactobacillus alimentarius, Lactobacillus curvatus, Lactobacillus casei subsp. casei, Lactobacillus sake, Lactococcus lactis, Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus carnosus, Staphylococcus xylosus, Saccharomyces (cerevisiae or also boulardii), Bacillus (cereus var toyo or subtilis), Bacillus coagulans, Bacillus licheniformis, Escherichia coli strain nissle, Propionibacterium freudenreichii and their mixtures.

12. The method according to claim 1, in which the microorganism results from the group of the lactic bacteria.

13. The method according to claim 1, in which the microorganism is of the species Lactobacillus paracasei or one of its fractions.

14. The method according to claim 1, in which the microorganism is employed in a proportion of 0.00001 to 20% by weight, with respect to a total weight of the composition comprising it.

15. The method according to claim 1, in which the hesperidin and its derivatives are selected from the group consisting of hesperitin and hesperitin glucuronide.

16. The method according to claim 1, in which the effective amount of hesperidin or of one of its derivatives is employed in a proportion of 0.00001 to 20% by weight, with respect to the total weight of a composition comprising it.

17. The method according to claim 1, comprising administration of the combination by topical, oral or parenteral route.

18. The method according to claim 1, in which the dose of hesperidin or one of its derivatives is employed so that to administer between 100 mg and 1000 mg of hesperidin or one of its derivatives per person and per day.

19. Cosmetic and/or dermatological composition comprising, in a physiologically acceptable carrier, at least an effective amount of hesperidin or of one of its derivatives in combination with at least an effective amount of at least one microorganism or one of its fractions.
